(19) United States
(12) Patent Application Publication
(10) Pub. No.: US 2025/0132043 A1
(43) Pub. Date: Apr. 24, 2025

(54) GENERATING PERSONALIZED HEALTH ANALYSES OF HEALTH INFORMATION

(71) Applicant: Phi Health Sciences, Inc., Salt Lake City, UT (US)

(72) Inventor: Kirk Roy OUIMET, Highland, UT (US)

(21) Appl. No.: 18/491,632

(22) Filed: Oct. 20, 2023

(51) Int. Cl. G16H 50/30; G16H 10/60

(52) U.S. Cl. CPC G16H 50/30; G16H 10/60

(57) ABSTRACT

A health manager may receive health information from a user, the health information including current health data associated with the user and anonymized user identification information. A health manager may, based on the health information and the anonymized user identification information, access, from a secure health datastore, health history for the user. A health manager may, based on the health information and the health history, generate a contextual health query for a foundation model system. A health manager may provide the contextual health query as an input to the foundation model system. A health manager may receive, from the foundation model system, a health analysis for the user based on the contextual health query.

### BACKGROUND

Recent years have seen improvements in the development of artificial intelligence (AI) and machine learning (ML). In particular, AI and ML models are being trained on massively large datasets, including millions or billions of datapoints. The datasets may include any type of data, including text, images, videos, sensor information, any other type of data, and combinations thereof. These models are trained to identify patterns within the dataset. Based on the identified patterns, the models may be used to extrapolate correlations between an input and a requested output.

Healthcare professionals are trained to receive health information and provide a health analysis based on the health information. But healthcare professionals are busy, and engaging with a healthcare professional may be expensive and difficult for a person to fit into his or her schedule. In the field of healthcare and health information, AI models are trained to prepare correlations between a user's health information and other related health information, including diagnoses, treatments, remedies, and so forth. But a person's health information is sensitive, and many people are not comfortable sharing health information online for fear of the health information being shared in an unapproved manner. Indeed, the government has stringent laws surrounding the receipt and handling of healthcare information, including the Health Insurance Portability and Accountability Act (HIPAA), which may limit the amount of information accessible and usable by health analysis systems. Accordingly, there is a need for a mechanism to anonymously receive health information and provide a health analysis without immediately and directly engaging with a healthcare provider.

### BRIEF SUMMARY

In some aspects, the techniques described herein relate to a method. A health manager receives health information from a user. The health information includes current health data associated with the user and anonymized user identification information. Based on the health information and the anonymized user identification information, the health manager accesses, from a secure health datastore, health history for the user. Based on the health information and the health history, the health manager generates a contextual health query for a foundation model system. The health manager provides the contextual health query as an input to the foundation model system. The health manager receives, from the foundation model system, a health analysis for the user based on the contextual health query.

In some aspects, the techniques described herein relate to a method. A health manager receives health information for a user. The health manager anonymizes the health information. The health manager generates a contextual health query based on the anonymized health information. The health manager provides the contextual health query as an input to a foundation model system. The health manager receives, from the foundation model system, a health analysis based on the contextual health query. The health manager generates a presentation including a personalized health analysis based on the health analysis from the foundation model system.

In some aspects, the techniques described herein relate to a system, including: a processor and memory, the memory including instructions that, when accessed by the processor, cause the processor to: receive health information from a user, the health information including current health data associated with the user and anonymized user identification information; based on the health information and the anonymized user identification information, access, from a secure health datastore, health history for the user; based on the health information and the health history, generate a contextual health query for a foundation model system; provide the contextual health query as an input to the foundation model system; and receive, from the foundation model system, a health analysis for the user based on the contextual health query.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

The detailed description provides one or more embodiments with additional specificity and detail through the use of the accompanying drawings, as briefly described below.

FIG. 1 is a schematic representation of a health management system, according to at least one embodiment of the present disclosure.

FIG. 2 is a representation of a health management system, according to at least one embodiment of the present disclosure.

FIG. 3 is a string diagram of a health management system, according to at least one embodiment of the present disclosure.

FIG. 4 is a representation of a health management system, according to at least one embodiment of the present disclosure.

FIG. 5 is a schematic representation a health management system, according to at least one embodiment of the present disclosure.

FIG. 6 is a representation of a health management system, according to at least one embodiment of the present disclosure.

FIGS. 7-1 and 7-2 are representations of a user device 702 on which a health management application is running, according to at least one embodiment of the present disclosure.

FIG. 8 is a flowchart of a method for analyzing a user's health, according to at least one embodiment of the present disclosure.

FIG. 9 is a flowchart of a method for analyzing a user's health, according to at least one embodiment of the present disclosure.

FIG. 10 is a schematic representation of a computing system, according to at least one embodiment of the present disclosure.

### DETAILED DESCRIPTION

This disclosure relates to generating a health analysis of a user based on health information provided by a user. A health management system may receive health information, such as a health inquiry, a health status update, a description of symptoms, health information from a health tracking device, and so forth. Using the health information, the health manager may generate a contextual health query to be used as an input to a foundation model system. The foundation model system may include one or more foundation models, such as large-language models (LLMs), and the foundation model(s) may prepare a health analysis based on the contextual health query. The health analysis may include a diagnosis, a recommendation, or other analysis of the health query. The health management system may provide the health analysis to the user. In this manner, the user may receive an analysis of his or her health information.

In some embodiments, the health management system may access the user's health history from a secure healthcare datastore. The user's health information and the secure healthcare datastore may be secured using end-to-end encryption. For example, the health management system may receive anonymized health information from the user and/or the health management system may anonymize the health information when the health information is received. The health management system may access the health history from the secure health datastore to further contextualize the health query. For example, the health information provided by the user may include information related to a particular symptom, and the health history may include historical information related to the user's experience with the symptom. In some examples, the health information may include background medical information, such as allergies, current medications, height, weight, sex, other medical information, and combinations thereof. Providing at least a portion of the user's health history may help to improve the health query and the associated health analysis from the foundation model system.

In some embodiments, the health manager may engage with a conversation with the user. For example, the health manager, when generating the health query, may request additional health information from the user. In some examples, the foundation model(s) from the foundation model system may request additional information from the user, and the health manager may request the additional information from the user. In some examples, the health analysis may cause the user to ask additional questions and/or provide additional information. In this manner, the user may enter a “chat” or provide additional information to further refine the health query and/or provide additional information regarding the health query.

The health management system provides many advantages and benefits over conventional systems and methods. For example, by anonymizing health information and anonymously accessing the secure health datastore, the health management system may help to improve health analyses for users by utilizing anonymized health information and/or health history from the user. Anonymizing health information utilizing end-to-end encryption may result in the foundation model(s) utilizing additional health information. This may result in increased relevance and/or accuracy in the health analysis of the health information.

In some embodiments, the health management system described herein may help to improve the aggregation and analysis of multiple foundation models. For example, the health management system described herein may submit the health query to multiple foundation models. Each foundation model may be trained using different parameters and/or on a different training database. The foundation models may each generate a different health analysis. The health management system may process the received health analyses and prepare a personalized health analysis for the user. The personalized health analysis may include any processing of the health analyses, including an aggregation of the analyses, a ranking of the analysis, follow-up questions, dismissal of irrelevant results, any other processing, and combinations thereof.

As illustrated by the foregoing discussion, the present disclosure utilizes a variety of terms to describe features and advantages of the health management system. Additional detail is now provided regarding the meaning of such terms. For example, as used herein, the term “health information” refers to information related to the user. In particular, the term “health information” can include physiological information, emotional information, demographical information, any other health information, and combinations thereof. To illustrate, health information may include quantitative measured physiological parameters, such as heart rate, blood pressure, temperature, electrical signals, blood type, blood chemistry, height, weight, sex, any other physiological information, and combinations thereof. In some examples, health information may include qualitative information, such as pain information, such as pain level, pain location, pain description, any other pain description, and combinations thereof. In some examples, health information may include other qualitative information, such as headaches, stomach aches, faintness, dizziness, light-headedness, increased sensitivity of one or more senses (e.g., sight, sound, tough, taste, smell, temperature), any other qualitative information, and combinations thereof. In some examples, health information may include emotional information, such as happiness, anger, sadness, guilt, any other emotional information, and combinations thereof. In some examples, health information may include mental-health information, such as depression, anxiety, confusion, social pressure, any other mental-health information, and combinations thereof. In some examples, health information may include demographic information, such as age, race, residence, travel locations, marital status, sexual activity, any other demographic information, and combinations thereof. In some embodiments, health information may include lifestyle information, such as diet, activity level, preferred activity types, any other lifestyle information, and combinations thereof.

As used herein, current health data may include health information provided by the user representative of the user's current health status. For example, current health data may include health information that is not included in any health datastore. In some examples, current health data may include new health information not previously received by the health management system.

As used herein, the term “anonymous” refers to the treatment of information without user-identifying information. For example, anonymous may refer to information in which identifying information has been removed and/or encrypted. User-identifying information may include name, address, social security number, any other user-identifying information, and combinations thereof. In some embodiments, health information may be anonymized. For example, health information may be anonymized using end-to-end encryption, such that the health management system has no access to user-identifying information. In some embodiments, user-identifying information may be anonymized, and a user may receive an anonymous user ID.

As used herein, the term “secure health datastore” refers to storage in which a user's health information and/or health history are stored. The secure health datastore may be an encrypted datastore. The encrypted datastore may prevent access to any person that does not have the appropriate key. The encrypted datastore may be encrypted and/or configured to comply with the relevant laws and regulations, including HIPAA laws and similar laws in other jurisdictions.

As used herein, the term “health history” refers to historical health information for a user. The historical health information may include any health information that has been previously provided and/or measured. The health history may be stored in the secure health datastore. In some embodiments, at least a portion of the health history may be collected by the health management system discussed herein. In some embodiments, at least a portion of the health history may be collected by third-parties, such as healthcare providers, insurance providers, medical devices, smart devices, any other third-party, and combinations thereof.

As used herein, the term “foundation model” refers to an AI or ML model that is trained to generate an output in response to an input based on a large dataset. In some embodiments, a foundation model may include a “large-language model (LLM),” although other foundation models may be utilized. A foundation model may include a neural network having a large number of parameters (e.g., billions of parameters) that the model may consider in performing a task or otherwise generating an output based on an input. In one or more embodiments described herein, a foundation model is trained to generate a response to a query. In some implementations, a foundation model refers to a health analysis model. The foundation model be trained on datasets of health information and/or diagnostic information. For example, the foundation model may be trained based on a set of health information and associated diagnoses of diseases and/or conditions. The foundation model may be trained to generate a health analysis of a user's input health information. The health analysis may include any analysis of the health information, including a diagnosis, recommended physical and/or mental activities, recommended new and/or adjustments to existing medications, recommended new and/or adjustments to existing nutrition supplements, any other analysis, and combinations thereof.

A foundation model may be a part of a foundation model system. For example, a foundation model system may include multiple foundation models. In some embodiments, the different foundation models may be trained using different datasets. In some embodiments, the different foundation models may be trained using different parameters and/or combination of parameters. In some embodiments, based on the different training datasets and/or parameters, the different foundation models may generate different responses to the same input.

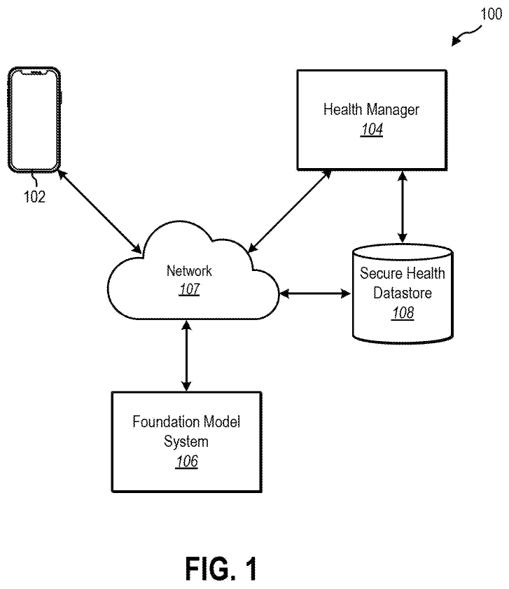

FIG. 1 is a schematic representation of a health management system 100, according to at least one embodiment of the present disclosure. A user may enter health information on a user device 102. A health manager 104 may receive the user's health information. The health manager 104 may generate a health query to submit to a foundation model system 106. The foundation model system 106 may prepare a health analysis based on the health query. The user may then receive the health analysis at the user device 102.

In some embodiments, the user device 102, health manager 104, and foundation model system 106 may be in communication over a network 107, such as the Internet. For example, the user device 102 may be in communication with the health manager 104 over the network 107. In some examples, the health manager 104 may be located on the user device 102. In some embodiments, the health manager 104 may be part of a server located remote from the user device 102.

The foundation model system 106 may be in communication with the health manager 104 and/or the user device 102 over the network 107. For example, the health manager 104 may generate health queries and provide the health query to the foundation model system 106 over the network 107. In some embodiments, the health manager 104 may be located on a different server than the foundation model system 106. In some embodiments, the health manager 104 may be located on the same server as the foundation model system 106. As discussed in further detail herein, the health manager 104 may be in communication with a secure health datastore 108. In some embodiments, the secure health datastore 108 may be part of the health manager 104. For example, the health manager 104 may maintain and/or update the secure health datastore 108 during operation. In some examples, the secure health datastore 108 may be connected to the network 107 and the health manager 104 may access the secure health information in the secure health datastore 108 over the network 107. In some examples, the secure health datastore 108 may be in communication with the user device 102 (for updates based on measured health information) over the network 107.

The user device 102 may include any type of device. For example, the user device 102 may include a computing device and the user may enter health information into the computing device. Such computing devices may include smartphones, tablets, computers, laptops, smart watches, any other computing device, and combinations thereof. In some examples, the user device 102 may include a medical device including one or more health sensors, such as an electrocardiogram (EKG), a pulse oximeter, a blood pressure sensor, a pulse monitor, an imaging device (e.g., camera, x-ray, infrared, CT scan, PET scan, nuclear imaging), any other medical device, and combinations thereof. In some embodiments, the user device 102 may include a wearable device that may collect health information, such as a wearable smart device, including smart watches, pedometers, rings, bracelets, necklaces, anklets, any other wearable smart device, and combinations thereof.

In some embodiments, the user may enter health information into a field in an application on the user device 102. For example, the user may open a chat application on the user device 102, and the user may enter one or more pieces of health information, such as a current status, symptom, or other health information. In some embodiments, the user device 102 may automatically provide measured health information to the health manager 104. For example, the user device 102 may include one or more health sensors, and the measured health information may be provided to the health manager 104.

The measured health information may be provided to the health manager 104 periodically and/or episodically. For example, the measured health information may be provided to the health manager 104 on a schedule, such as every 1 second, 30 seconds, 1 minute, 30 minutes, hour, 6 hours, 12 hours, 1 day, 2 days, 3 days, 5 days, week, 2 weeks, 1 month, 3 months, 6 months, 1 year, periods greater than one year, and combinations thereof. In some examples, the measured health information may be provided to the health manager 104 episodically, or based on pre-determined events. Such pre-determined events may include upon request by the user device 102 and/or the health manager 104, based on abnormal sensor measurements, upon capture of the health information, based on a recommendation by a health-care professional, any other episodic event, and combinations thereof.

When the health manager 104 receives the health information from the user, the health manager 104 may generate a health query to submit to the foundation model system 106. In some embodiments, the health manager 104 may generate a health query to the foundation model system 106 when the health manager 104 receives the health information. In some embodiments, the health manager 104 may generate a health query when the user requests a health analysis. In some embodiments, the health manager 104 may generate a health query when the user enters free-form health information into the health app on the user device 102.

The health manager 104 may generate a contextual health query. The contextual health query may include context information to improve the accuracy and/or relevance of the results from the foundation model system 106. For example, the contextual health query may include context related to the health information provided by the user. Such context may include related symptoms, a health database for the foundation model system 106 to consider, a diagnosis database for the foundation model system 106 to consider, follow-up questions answered by the user, any other context information, and combinations thereof.

In some embodiments, the health manager 104 may access a secure health datastore 108 to generate the health query. For example, the health manager 104 may access the secure health datastore 108 to generate context for a contextual health query. In some examples, the health manager 104 may access a user profile in the secure health datastore 108 to access health history for the user. The health history accessed by the health manager 104 from the secure health datastore 108 may provide context for the health query. In some embodiments, the health manager 104 may access related health history to the health information provided, including health information related to the same or similar symptoms, question, or status provided by the user. In some embodiments, the health manager 104 may access health history that is seemingly unrelated to the health information provided by the user. The seemingly unrelated information may provide context to the health query and allow the foundation model system 106 to generate correlations between the health history and the health information. This may help to improve the relevance and/or accuracy of the health analysis provided by the foundation model system 106.

In some embodiments, the contextual health query may be generated into a format that is tailored to the particular foundation model(s) in the foundation model system 106. For example, different foundation models may utilize different data input formats and/or recognize patterns in data differently based on the format in which the data is received by the foundation model. By generating a contextual health query having context that is tailored to a particular foundation model, the relevance and/or accuracy of the health analysis may be improved. In some embodiments, the health manager 104 may generate a single contextual health query for each foundation model in a multi-model foundation model system 106. In some embodiments, the health manager 104 may generate different contextual health queries for each different foundation model in the foundation model system 106.

In some embodiments, as discussed in further detail herein, the health management system 100 may include a chatbot or other feedback mechanism. For example, the application on the user device 102 may include a window in which the user may enter text, and the health manager 104 and/or the foundation model system 106 may engage in communication with the user device 102 through a series of messages. In some examples, the foundation model system 106 and/or the health manager 104 may include natural language processing to present the health analysis and/or ask for additional information using natural language. In some examples, the foundation model system 106 and/or the health manager 104 may request additional information in the form of a follow-up question. This may help to improve engagement by the user, thereby improving the health information gathered by the user.

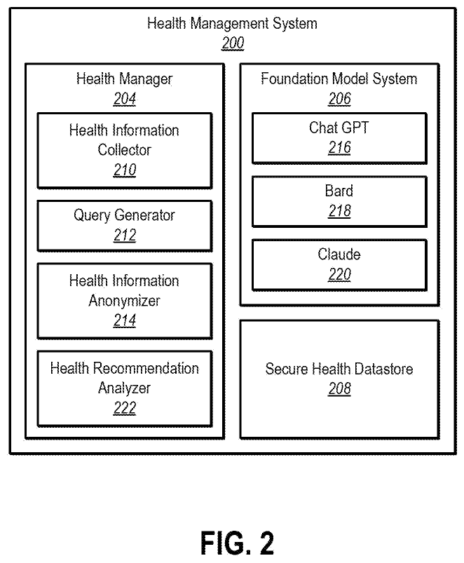

FIG. 2 is a representation of a health management system 200, according to at least one embodiment of the present disclosure. Each of the components of the health management system 200 can include software, hardware, or both. For example, the components can include one or more instructions stored on a computer-readable storage medium and executable by processors of one or more computing devices, such as a client device or server device. When executed by the one or more processors, the computer-executable instructions of the health management system 200 can cause the computing device(s) to perform the methods described herein. Alternatively, the components can include hardware, such as a special-purpose processing device to perform a certain function or group of functions. Alternatively, the components of the health management system 200 can include a combination of computer-executable instructions and hardware.

Furthermore, the components of the health management system 200 may, for example, be implemented as one or more operating systems, as one or more stand-alone applications, as one or more modules of an application, as one or more plug-ins, as one or more library functions or functions that may be called by other applications, and/or as a cloud-computing model. Thus, the components may be implemented as a stand-alone application, such as a desktop or mobile application. Furthermore, the components may be implemented as one or more web-based applications hosted on a remote server. The components may also be implemented in a suite of mobile device applications or “apps.”

The health management system 200 includes a health manager 204. The health manager may include a health information collector 210. The health information collector 210 may collect information from the user. For example, the health information collector 210 may receive text input from the user, measured health information from one or more health sensors, any other health information, and combinations thereof.

The health manager 204 may further include a query generator 212. The query generator 212 may generate a contextual health query based on the health information collected by the health information collector 210. In some embodiments, as discussed in further detail herein, the query generator 212 may generate the contextual health query using health history information accessed from a secure health datastore 208.

The health manager 204 may include a health information anonymizer 214 that may anonymize the health information received from the user. In some embodiments, the anonymizer 214 may anonymize the health information at the health manager 204. For example, the health information collector 210 may receive the health information from the user including any identifying information. When the health information collector 210 receives the health information, the health information anonymizer 214 may anonymize the health information. The health information anonymizer 214 may anonymize the health information in any manner. For example, the health information anonymizer 214 may anonymize the health information by encrypting the user-identifying information in the files. In some examples, the health information anonymizer 214 may anonymize the health information by deleting the user-identifying information from the files. In some embodiments, the health information anonymizer 214 may anonymize the health information at the user device. In this manner, the health information collector 210 may not receive and/or have access to any user-identifying information.

In some embodiments, the health manager 204 may update the secure health datastore 208 with the user's health information. For example, the health information collector 210 may receive health information that is not stored in the secure health datastore 208. The health information anonymizer 214 may determine that the health information received from the user is not stored in the secure health datastore 208. When the health manager 204 determines that the health information is not stored in the secure health datastore 208, the health manager 204 may store the health information in the secure health datastore 208. In some embodiments, the health manager 204 may update the user's profile with the new health information. In some embodiments, the health manager 204 may anonymously update the user's profile in the secure health datastore 208 with the new health information.

The health manager 204 may provide, as an input, the contextual health query to a foundation model system 206. The foundation model system 206 may analyze the contextual health query and provide a health analysis to the health manager 204. As discussed herein, the foundation model system 206 may include one or more foundation models. In some embodiments, the foundation model system 206 may include commercial foundation models. Some non-limiting examples of foundation models may include ChatGPT 216, Bard 218, and Claude 220. While specific types of foundation models are provided herein, including specific LLMs, it should be understood that the foundation model system 206 may include any foundation model, including other types of foundation models, foundation models trained using images, foundation models trained using health sensor data, foundation models yet-to-be developed, any other foundation model, and combinations thereof.

The foundation model system 206 may analyze the contextual health query and provide a health analysis. For example, the foundation model system 206 may prepare an identification of a medical issue, a diagnosis, a recommendation, any other analysis, and combinations thereof. The foundation model system 206 may provide the health analysis to a health analysis analyzer 222. The health analysis analyzer 222 may process the health analysis and prepare a presentation for the user. The health analysis analyzer 222 may prepare a presentation that includes any type of information, such as text information, visual information, haptic information, audible information, any other type of information, and combinations thereof.

In some embodiments, each of the foundation models of the foundation model system 206 may separately process the contextual health query. For example, the foundation model system 206 may provide the contextual health query as input to each of the foundation models in the foundation model system 206. Each of the foundation models may prepare a health analysis, and the foundation model system 206 may provide each of the health analyses to the health manager 204. In some embodiments, the foundation models in the foundation model system 206 may analyze the health analysis of one or more of the other foundation models in the foundation model system 206. This cross-checking may help to improve the accuracy and/or relevance of the results of the health analysis.

The health analysis analyzer 222 may receive and process the multiple health analyses from the foundation model system 206. For example, the health analysis analyzer 222 may aggregate the health analyses from the foundation model system 206 to generate a personalized health analysis for the user. In some examples, the health analysis analyzer 222 may rank the health analyses of the foundation model system 206, such as by relevance, accuracy, detail, any other ranking, and combinations thereof. In some examples, the health analysis analyzer 222 may categorize the health analyses of the foundation model system 206, such as by type of analysis, diagnoses, recommendations, any other categorization, and combinations thereof.

In some embodiments, the foundation model system 206 and/or the health manager 204 may identify additional information that may improve the personalized health analysis. The additional information may include additional details about a symptom, recurrence of the symptoms, contextual information, additional details about other health information, any other additional information, and combinations thereof.

As discussed herein, the health analysis analyzer 222 may generate a personalized health analysis for the user. The health analysis analyzer 222 may provide the personalized health analysis to the user, such as in the form of a presentation, as discussed herein. In some embodiments, the user may, based on the presentation of the personalized health analysis, provide additional detail to the health manager 204 and/or provide the health manager 204 with additional questions. This process may be iterative, forming a conversation between the user and the health management system 200.

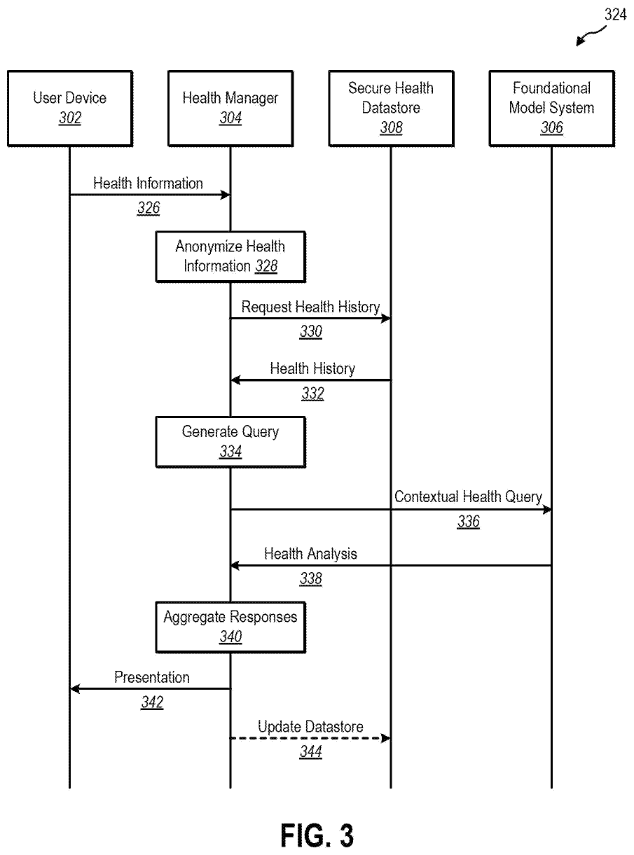

FIG. 3 is a string diagram 324 of a health management system, according to at least one embodiment of the present disclosure. A user may interact with a user device 302 to provide health information 326 to a health manager 304. The health manager 304 may anonymize 328 the health information. As discussed herein, the health management system may utilize end-to-end encryption to maintain security and privacy of the user's health information 326. In some embodiments, the anonymization may occur at the user device 302. In this manner, the health information 326 received by the health manager 304 may already be anonymized when the health manager 304 processes the health information 326. In some embodiments, the anonymization may occur when the health manager 304 receives the health information 326.

The health manager 304 may send a request 330 for health history for the user from a secure health datastore 308. The secure health datastore 308 may provide 332 the health history for the user to the health manager 304. In some embodiments, the request 330 may include a request for specific health history, such as health history related to the health information 326. In some embodiments, the request 330 may include a request for health history from a specific period of time, such as the time during which the health information 326 was recorded, health history for a period of time before the health information 326 was provided to the health manager 304, health history for a period of time identified in the health information 326, health history for the entire duration stored in the secure health datastore 308, any other period of time, and combinations thereof. In some embodiments, the secure health datastore 308 may provide all of the stored health history to the user.

In some embodiments, the anonymization may the user's data may occur before requesting the health history from the secure health datastore 308. For example, the user identification information may be anonymized into an anonymous user ID, and the health history in the secure health datastore 308 may be stored under the same anonymous user ID. In this manner, the secure health datastore 308 may not include any user-identifying information. In some embodiments, the health information 326 and/or health history 332 may be anonymized after receiving the health history 332. For example, the health manager 304 may receive the health information 326 including user-identifying information. The health manager 304 may receive the health history 332 having user-identifying information. The health manager 304 may anonymize the health information 326 and the health history 332 after receiving the health history 332. In this manner, the secure health datastore 308 may store the health history 332 with user identifying information. This may allow the health manager 304 to access third-party databases.

Using the health information 326 and/or the health history 332, the health manager 304 may generate 334 a contextual health query 336. The health manager 304 may send the contextual health query 336 to a foundation model system 306. The foundation model system 306 may process the contextual health query 336 and generate one or more health analyses 338.

The health manager 304 may aggregate 340 the health analyses 338. For example, the health manager 304 may rank, categorize, summarize, or otherwise aggregate 340 the one or more health analyses 338. The health manager 304 may prepare a presentation 342 with the aggregated health analyses 338. The user may review the presentation 342 on the user device 302.

In some embodiments, the health manager 304 may update 344 the secure health datastore 308. For example, the health manager 304 may update the secure health datastore 308 with the health information 326 provided by the user device 302. In some examples, the health manager 304 may update the secure health datastore 308 with at least a portion of the health analyses 338. In some embodiments, the health manager 304 may update the secure health datastore 308 with the presentation 342. In some embodiments, the health manager 304 may update the secure health datastore 308 with any relevant health information that is used and/or processed by the health management system. In some embodiments, the health manager 304 may update the secure health datastore 308 after providing the presentation 342 to the user. In some embodiments, the health manager 304 may update the secure health datastore 308 as soon as new health information is received. In some embodiments, the health manager 304 may update the secure health datastore 308 with the request 330 for the health history 332.

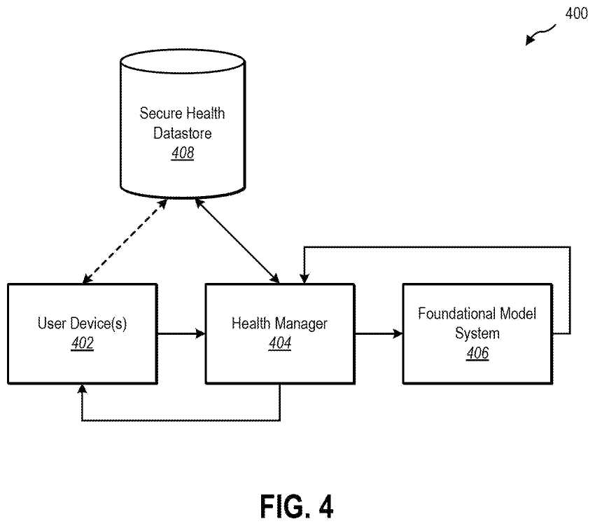

FIG. 4 is a representation of a health management system 400, according to at least one embodiment of the present disclosure. A user device 402 may provide a health manager 404 with health information. As discussed herein, the user device 402 may include any number of devices, including computing devices, health sensors, wearable smart devices, any other device, and combinations thereof. In some embodiments, the user device 402 may provide a secure health datastore 408 with the health information.

As discussed herein, the health manager 404 may receive the health information and generate a health query. In some embodiments, the health manager 404 may receive health history for the user from the secure health datastore 408. The health manager 404 may generate a contextual health query using the health information and the health history.

The health manager 404 may provide the contextual health query to a foundation model system 406. The foundation model system 406 may receive the contextual health query as input and process the contextual health query based on their training. In some embodiments, the foundation model system 406 may provide the health analysis to the health manager 404. In some embodiments, the foundation model system 406 may provide a request for more information. In some embodiments, the health manager 404 may identify the additional requested information as health history accessible at the secure health datastore 408. The health manager 404 may retrieve the health history from the secure health datastore 408 and provide the additional health history to the foundation model system 406.

In some embodiments, the foundation model system 406 and/or the health manager 404 may request additional health information from the user. The health manager 404 may generate a presentation or other feedback to request additional health information from the user. For example, the user device 402 may include a chat application, and the health manager 404 may generate a chat response with the user requesting additional information. The user may provide the additional health information to the health manager 404, which may provide the additional health information to the foundation model system 406. In this manner, the health management system 400 may engage in a conversation and/or a feedback loop with the user.

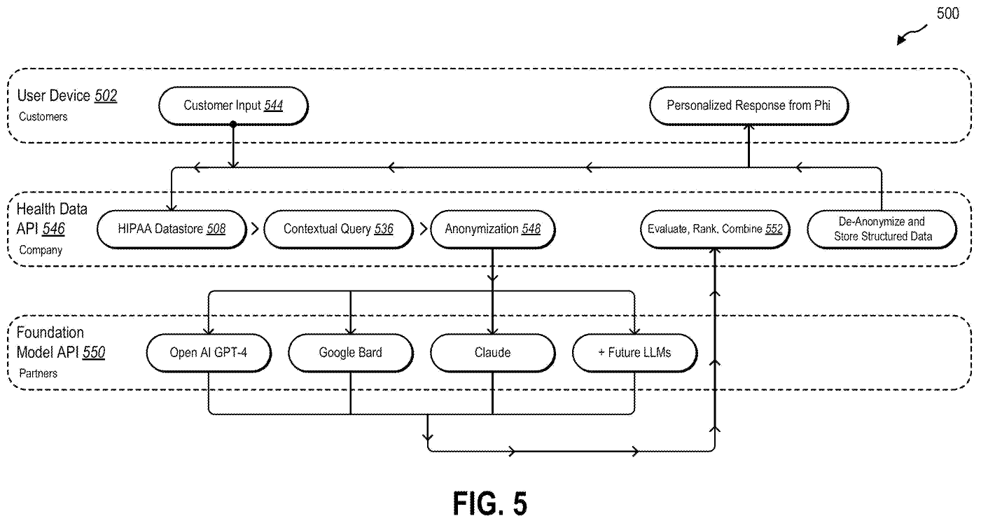

FIG. 5 is a schematic representation a health management system 500, according to at least one embodiment of the present disclosure. The embodiment shown in health management system 500 is a specific, non-limiting example, and the skilled artisan will understand that the techniques described herein may be applied to other examples and embodiments. A user device 502 may receive user input 544. As discussed herein, the customer input 544 may be any type of user input, including text input, health sensor measurements, any other user input, and combinations thereof. A health data application programming interface (API) 546 may receive the user input 544. In some embodiments, the health data API 546 may be the health managers discussed herein (e.g., the health manager 104, health manager 204, health manager 304, health manager 404). In some embodiments, the health data API 546 may be any other API configured to perform the acts described herein.

The health data API 546 may include and/or access a secure health datastore 508, which may include a health history for the user. The health data API 546 may generate a contextual health query 536. In the embodiment shown, the health data API 546 may anonymize 548 the contextual health query 536. However, as discussed in further detail herein, the user input, health history, and other user-identifying information may be anonymized at any point in time along the information flow chart.

The anonymized health query may be provided to a foundation model API 550. The foundation model API 550 may include multiple foundation models. The anonymized health query may provide to each of the foundation models in the foundation model API 550. The foundation model API 550 may provide the health analyses generated by the foundation models to the health data API 546.

The health data API 546 may process 552 the health analyses, such as by evaluating, ranking, and/or combining the health analyses. The health data API 546 may de-anonymize 554 the health analyses and provide 556 the personalized health analyses to the user device 502. In some embodiments, the health data API 546 may update the secure health datastore 508 with the personalized health analyses. This process may be repeated through follow-up by the user and/or using new health information provided by the user.

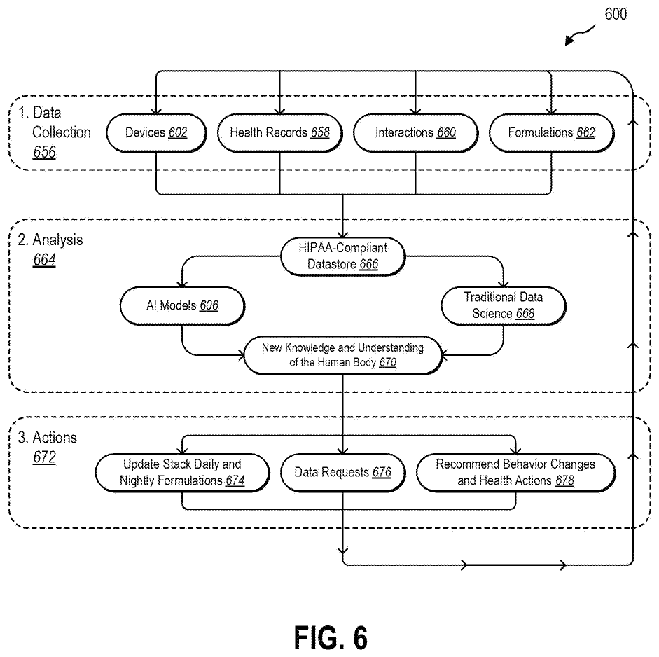

FIG. 6 is a representation of a health management system 600, according to at least one embodiment of the present disclosure. The health management system 600 shown includes different stages. In a data collection stage 656, the health management system 600 may collect health information from one or more sources. For example, the health management system 600 may collect health information from one or more user devices 602. The health management system 600 may further collect health information from health records 658, such as blood tests, DNA samples, MRI scans, X-rays, EKG/ECG results, mammograms, allergy tests, any other health record 658, and combinations thereof. The health management system 600 may further collect health information from interactions 660 with an application. The interactions may take the form of declarative statements (e.g., “I have a headache,” “I keep waking up at night,” “My psoriasis is acting up,” “I'm fatigued right now,” “My eyes are itching,” “My stomach hurts,” “I can't think clearly”), user-provided information (e.g., “Here is a picture of my psoriasis”), questions (“Why do I hurt in my throat?”), any other interaction, and combinations thereof. In some embodiments, the health management system 600 may further collect health information regarding supplements and medications 662 the user is taking, including the quantities of particular vitamins, minerals, drugs, and so forth.

In an analysis stage 664, the health information collected during the data collection stage 656 may be analyzed, such as by a health manager, a health data API, or other analysis tool. In the analysis stage 664, the health information may be stored in a secure health datastore 608. The secure health datastore 608 may further include health history for the user. The health information and/or health history may be provided to a foundation model system 606, where one or more foundation models may process the health information and/or the health history. In some embodiments, the health information and/or the health history may be analyzed using one or more conventional analysis techniques 668, such as a human doctor analysis. The analyzed health information and/or health history may facilitate new knowledge and/or understanding of the human body 670.

The analysis of the health information and/or health history may result in the user taking one or more actions in an action stage 672. In some embodiments, the analysis may include the actions to be taken. In some embodiments, the user may take the actions in the action stage 672 of his or her own accord. For example, the user may update his or her medication and/or supplements at 674. In some examples, the action may include requests 676 for more information. In some examples, the action may include recommended behavior changes and/or health actions 768.

After and/or while performing the actions outlined in the action stage 672, the user's actions may be provided as information in the data collection stage 656. The health management system 600 may indefinitely loop through the stages shown. In this manner, the user may consistently receive updates to his or her health. This may help to increase the user's healthspan, or the length of time in which the user has a healthy body where the user can do the things he or she desires to do.

FIGS. 7-1 and 7-2 are representations of a user device 702 on which a health management application is running, according to at least one embodiment of the present disclosure. The user device 702 includes a graphical user interface (GUI) 770. In the GUI 770, the user may enter health information in an input field 772. For example, the input field 772 shown is a text input field, and the user has typed “I have a headache.” While text input has been shown, it should be understood that the input field 772 may receive any type of input, such as image input, input from the camera of the user device 702, sensor input, audible input, any other type of input, and combinations thereof.

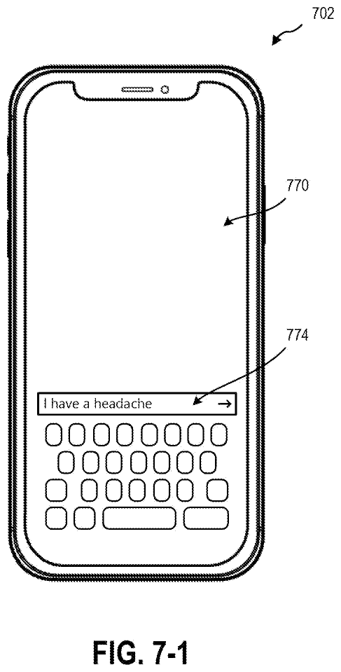

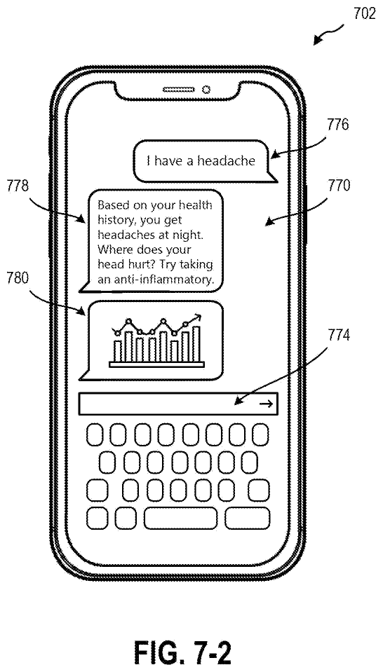

In the input field 772 shown, the user may begin a chat with the health manager. For example, after the health manager sends the health information to the foundation model system (using a contextual health query provided to one or more foundation models). The health manager may prepare a presentation as a response to send to the user. For example, as shown in FIG. 7-2, the user's message 776 is provided in a first bubble. The health manager may prepare a presentation 778 for the user. The presentation 778 may include any type of presentation. For example, the presentation 778 shown in FIG. 7-2 includes a text report to the user. The presentation 778 may include any type of information, such as an analysis of the health information, a request for more information, a recommendation or recommended activity, any other information, and combinations thereof.

In the embodiment shown, the presentation 778 includes a chart 780. The chart 780 may visually and/or graphically present the health analysis in a manner that is easily understandable by the user. In some embodiments, the presentation 778 may include any other media, such as GIFs, videos, infographics, podcasts, any other media, and combinations thereof. Preparing the presentation 778 may help to improve the communication and/or interaction of the user with the health manager.

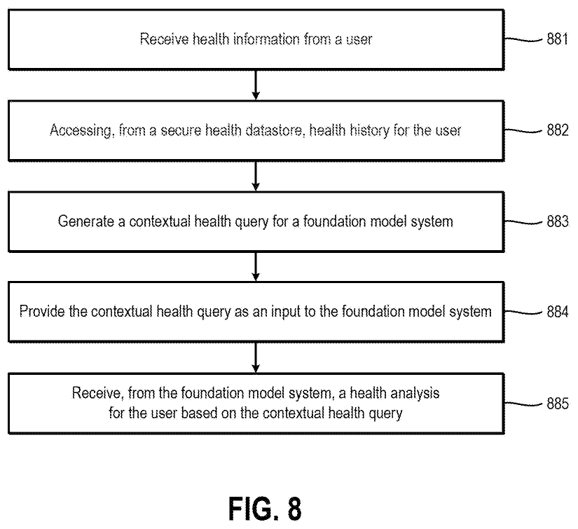

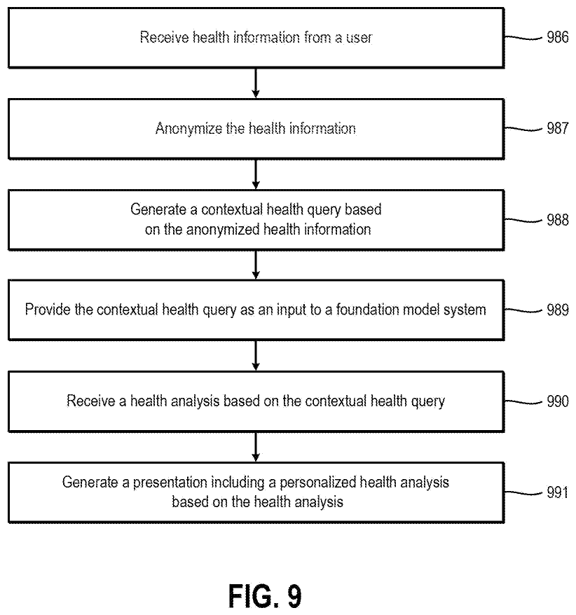

FIGS. 8 and 9, the corresponding text, and the examples provide a number of different methods, systems, devices, and computer-readable media of the health management system. In addition to the foregoing, one or more embodiments can also be described in terms of flowcharts comprising acts for accomplishing a particular result, as shown in FIGS. 8 and 9. FIGS. 8 and 9 may be performed with more or fewer acts. Further, the acts may be performed in differing orders. Additionally, the acts described herein may be repeated or performed in parallel with one another or parallel with different instances of the same or similar acts.

As mentioned, FIG. 8 illustrates a flowchart of a series of acts for analyzing health information, according to at least one embodiment of the present disclosure. While FIG. 8 illustrates acts according to one embodiment, alternative embodiments may omit, add to, reorder, and/or modify any of the acts shown in FIG. 8. The acts of FIG. 8 can be performed as part of a method. Alternatively, a computer-readable medium can comprise instructions that, when executed by one or more processors, cause a computing device to perform the acts of FIG. 8. In some embodiments, a system can perform the acts of FIG. 8.

A health manager may receive health information from a user at 881. The health information may include current health data associated with the user. For example, the health information may include current health data that is an update to the user's health information and/or an update based on the user's current health information. The health information may further include anonymized user identification information.

Based on the health information and the anonymized user identification information, the health manager may access, from secure health datastore, a health history for the user at 882. Based on the health information and the health history, the health manager may generate a contextual health query for a foundation model system at 883. The health manager may provide the contextual health query as an input to the foundation model system at 884. In some embodiments, prior to providing the contextual health query to as the input to the foundation model system, the health manager may anonymize the health data and the health data for the contextual health query. In some embodiments, the health manager may de-anonymize the health analysis and provide the de-anonymized health analysis to the user.

The health manager may receive, from the foundation model system, a health analysis for the user based on the contextual health query at 885. In some embodiments, the foundation model system may include a plurality of foundation models in the foundation model system. In some embodiments, the health manager may generate a personalized health analysis from the health analyses generated by the different foundation models. In some embodiments, generating the personalized health analysis may include combining at least two health analyses from the foundation models.

As mentioned, FIG. 9 illustrates a flowchart of a series of acts for analyzing health information, according to at least one embodiment of the present disclosure. While FIG. 9 illustrates acts according to one embodiment, alternative embodiments may omit, add to, reorder, and/or modify any of the acts shown in FIG. 9. The acts of FIG. 9 can be performed as part of a method. Alternatively, a computer-readable medium can comprise instructions that, when executed by one or more processors, cause a computing device to perform the acts of FIG. 9. In some embodiments, a system can perform the acts of FIG. 9.

A health manager may receive health information from a user at 986. The health manager may anonymize the health information at 987. In some embodiments, anonymizing the health information includes anonymizing the health information using end-to-end encryption. The health manger may generate a contextual health query based on the anonymized health information at 988. The health manager may provide the contextual health query as an input to a foundation model system at 989. The health manager may receive a health analysis based on the contextual health query at 990. The health manager may generate a presentation including a personalized health analysis based on the health analysis at 991. In some embodiments, the health analysis may include a health recommendation to adjust a health activity of the user. In some embodiments, the health recommendation includes adjusting a supplement stack of the user.

In some embodiments, the personalized health analysis includes a request for more health information from the user. The health manager may then receive additional health information from the user responsive to the request for more health information. The health manager may update the contextual health query with the additional health information and/or generate a new contextual health query based on the additional health information. The health manager may provide the updated contextual health query and/or the new contextual health query to as input to the foundation model system. The health manager may receive, from the foundation model system, an updated health analysis. The health manager may then provide the updated health analysis to the user. In this manner, the health manager may provide an interactive health conversation with the user.

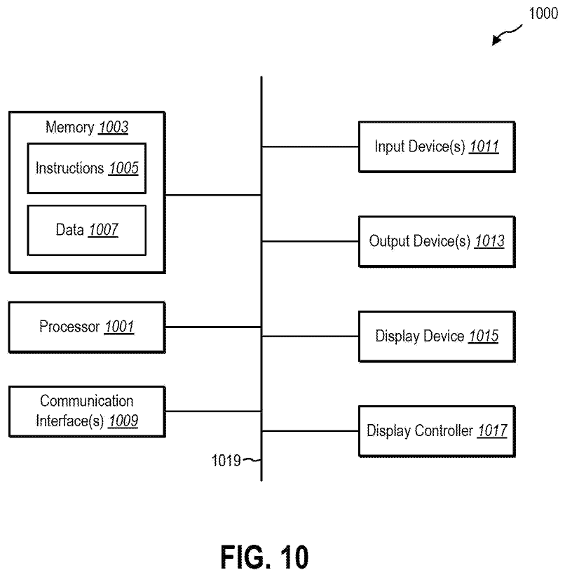

FIG. 10 illustrates certain components that may be included within a computer system 1000. One or more computer systems 1000 may be used to implement the various devices, components, and systems described herein.

The computer system 1000 includes a processor 1001. The processor 1001 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 1001 may be referred to as a central processing unit (CPU). Although just a single processor 1001 is shown in the computer system 1000 of FIG. 10, in an alternative configuration, a combination of processors (e.g., an ARM and DSP) could be used.

The computer system 1000 also includes memory 1003 in electronic communication with the processor 1001. The memory 1003 may be any electronic component capable of storing electronic information. For example, the memory 1003 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 1005 and data 1007 may be stored in the memory 1003. The instructions 1005 may be executable by the processor 1001 to implement some or all of the functionality disclosed herein. Executing the instructions 1005 may involve the use of the data 1007 that is stored in the memory 1003. Any of the various examples of modules and components described herein may be implemented, partially or wholly, as instructions 1005 stored in memory 1003 and executed by the processor 1001. Any of the various examples of data described herein may be among the data 1007 that is stored in memory 1003 and used during execution of the instructions 1005 by the processor 1001.

A computer system 1000 may also include one or more communication interfaces 1009 for communicating with other electronic devices. The communication interface(s) 1009 may be based on wired communication technology, wireless communication technology, or both. Some examples of communication interfaces 1009 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 1000 may also include one or more input devices 1011 and one or more output devices 1013. Some examples of input devices 1011 include a keyboard, mouse, microphone, remote control device, button, joystick, trackball, touchpad, and lightpen. Some examples of output devices 1013 include a speaker and a printer. One specific type of output device that is typically included in a computer system 1000 is a display device 1015. Display devices 1015 used with embodiments disclosed herein may utilize any suitable image projection technology, such as liquid crystal display (LCD), light-emitting diode (LED), gas plasma, electroluminescence, or the like. A display controller 1017 may also be provided, for converting data 1007 stored in the memory 1003 into text, graphics, and/or moving images (as appropriate) shown on the display device 1015.

The various components of the computer system 1000 may be coupled together by one or more buses, which may include a power bus, a control signal bus, a status signal bus, a data bus, etc. For the sake of clarity, the various buses are illustrated in FIG. 10 as a bus system 1019.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A method, comprising:
receiving health information from a user, the health information including current health data associated with the user and anonymized user identification information;
based on the health information and the anonymized user identification information, accessing, from a secure health datastore, health history for the user;
based on the health information and the health history, generating a contextual health query for a foundation model system;
providing the contextual health query as an input to the foundation model system; and
receiving, from the foundation model system, a health analysis for the user based on the contextual health query.

2. The method of claim 1, further comprising, prior to providing the contextual health query to the foundation model system, anonymizing the health information and the health history included in the contextual health query.

3. The method of claim 2, further comprising:
de-anonymizing the health analysis; and
providing the health analysis to the user.

4. The method of claim 1, wherein the foundation model system includes a plurality of foundation models and wherein receiving the health analysis includes receiving a plurality of health analyses from the plurality of foundation models in the foundation model system.

5. The method of claim 4, further comprising generating a personalized health analysis based on the plurality of health analyses.

6. The method of claim 5, wherein generating the personalized health analysis includes combining at least two health analyses of the plurality of health analyses.

7. The method of claim 5, wherein generating the personalized health analysis includes ranking the plurality of health analyses.

8. The method of claim 1, further comprising updating the health history in the secure health datastore with at least one of the contextual health query or the health information in the secure health datastore.

9. A method, comprising:
receiving health information for a user;
anonymizing the health information;
generating a contextual health query based on the anonymized health information;
providing the contextual health query as an input to a foundation model system;
receiving, from the foundation model system, a health analysis based on the contextual health query; and
generating a presentation including a personalized health analysis based on the health analysis from the foundation model system.

10. The method of claim 9, wherein anonymizing the health information includes anonymizing the health information using end-to-end encryption.

11. The method of claim 9, wherein the personalized health analysis includes a health recommendation to adjust a health activity for the user.

12. The method of claim 11, wherein the health recommendation includes an adjustment to a supplement stack.

13. The method of claim 9, wherein the personalized health analysis includes a request for more health information from the user, and further comprising:
receiving additional health information from the user responsive to the request;
updating the contextual health query with the additional health information;
provide the updated contextual health query as the input to the foundation model system;
receiving, from the foundation model system, an updated health analysis; and
providing the updated health analysis to the user.

14. A system, comprising:
a processor and memory, the memory including instructions that, when accessed by the processor, cause the processor to:
receive health information from a user, the health information including current health data associated with the user and anonymized user identification information;
based on the health information and the anonymized user identification information, access, from a secure health datastore, health history for the user;
based on the health information and the health history, generate a contextual health query for a foundation model system;
provide the contextual health query as an input to the foundation model system; and
receive, from the foundation model system, a health analysis for the user based on the contextual health query.

15. The system of claim 14, wherein the instructions further cause the processor to, prior to providing the contextual health query to the foundation model system, anonymize the health information and the health history included in the contextual health query.

16. The method of claim 15, wherein the instructions further cause the processor to:
de-anonymize the health analysis; and
provide the health analysis to the user.

17. The method of claim 14, wherein the foundation model system includes a plurality of foundation models and wherein receiving the health analysis includes receiving a plurality of health analyses from the plurality of foundation models in the foundation model system.

18. The method of claim 17, wherein the instructions further cause the processor to generate a personalized health analysis based on the plurality of health analyses.

19. The method of claim 18, wherein generating the personalized health analysis includes combining at least two health analyses of the plurality of health analyses.

20. The method of claim 18, wherein generating the personalized health analysis includes ranking the plurality of health analyses.
